(19) United States
(12) Patent Application Publication
(10) Pub. No.: US 2015/0174084 A1
(43) Pub. Date: Jun. 25, 2015

(54) Stable Liquid Medication Containing Diphenhydramine

(71) Applicant: The Procter & Gamble Company, Cincinnati, OH (US)

(72) Inventors: Allyn Michael Kaufmann, Morrow, OH (US); Roger William Krummen, Cincinnati, OH (US); Ashraf Traboulsi, West Chester, OH (US); Thomas Edward Huetter, West Chester, OH (US)

(21) Appl. No.: 14/576,260

(22) Filed: Dec. 19, 2014

Related U.S. Application Data
(60) Provisional application No. 61/918,687, filed on Dec. 20, 2013.

(51) Int. Cl. A61K 31/138

(52) U.S. Cl. CPC A61K 31/138

(57) ABSTRACT

A chemically stable liquid medication that contains diphenhydramine hydrochloride and has a pH of greater than about 4.5. The liquid medication contains less than about 1.5% of BZH based on parent DPH after 14 days at 75° C. according to the Stability Prediction Method. The liquid medication is adapted for consumption by adults and children 12 years and over.

### FIELD OF THE INVENTION

The present invention is directed towards medication, more particularly a liquid medication that comprises diphenhydramine.

### BACKGROUND OF THE INVENTION

Diphenhydramine hydrochloride (DPH) is a common active in over-the-counter medication used to treat allergic symptoms including itchiness, insomnia, motion sickness and extrapyramidal symptoms. DPH can be in both solid and liquid medications. However, it can be difficult to formulate a stable liquid DPH medication where both the color and DPH are stable over time.

Liquid medications are bottled and stored for a considerable period of time. However, if the solution does not have the correct properties, DPH can degrade, shortening the shelf life of the product.

Furthermore, DPH and colorants can degrade when exposed to heat, such as warm temperatures that may be encountered during shipping, handling, and storage. Reducing sugars, like those found in high-fructose corn syrup (HFCS), can “brown” via the Malliard reaction with the addition of heat thereby changing the product color.

Also, when DPH and a colorant are combined in a liquid medication, many colorants can degrade when exposed to ultraviolet (UV) light. Current regulations, such as the United States Pharmacopeia (USP), require that liquid DPH products be sold in cardboard boxes, dark colored or opaque bottles, and/or bottles with a UV-inhibitor that limits the amount of UV light that passes through. These packaging requirements can increase packaging cost and the amount of packaging, reduce the aesthetic appearance of the product, and can make it difficult for a consumer to tell how much medication is left in the bottle during use.

As such, there remains a need for a stable liquid medication that contains DPH and optionally a colorant. Furthermore, there is a need for a liquid medication that is chemically stable and color stable in UV light and heat and can be packaged in a translucent, colorless bottle without a UV-inhibitor.

### SUMMARY OF THE INVENTION

A chemically stable liquid medication comprising diphenhydramine hydrochloride and a pH of greater than about 4.5 and wherein the liquid medication comprises less than about 1.5% of BZH based on parent DPH after 14 days at 75° C. according to the Stability Prediction Method and wherein the liquid medication is adapted for consumption by adults and children 12 years and over.

A color stable liquid medication comprising diphenydramine and a pH of greater than about 4.0 wherein a color change is not visually perceptible and wherein the medication is substantially free of high fructose corn syrup.

A chemically stable liquid medication comprising diphenhydramine hydrochloride and a pH of greater than about 4.5 and wherein the liquid medication is contained in a translucent container and wherein the container does not have a UV-inhibitor and no secondary container is required for stability.

### BRIEF DESCRIPTION OF THE DRAWINGS

The patent or application file contains at least one photograph executed in color. Copies of this patent or patent application publication with color photograph(s) will be provided by the Office upon request and payment of the necessary fee.

While the specification concludes with claims particularly pointing out and distinctly claiming the subject matter of the present invention, it is believed that the invention can be more readily understood from the following description taken in connection with the accompanying drawings, in which:

FIG. 1 shows the dependence of benzhyrol (BZH) formation as a function of temperature at 14 days for Examples 1-5 and two currently available products;

FIG. 2 shows a model for the predicted percent degradation in Examples 1, 2, 4, 5, and 7 and two currently available products after a period of 24 months;

FIG. 3 shows the color shift for Examples 3, 6, and 8;

FIG. 4A shows the magnitude of the color shift for Example 3;

FIG. 4B shows the magnitude of the color shift for Example 6;

FIG. 4C shows the magnitude of the color shift for Example 2;

FIG. 4D shows the magnitude of the color shift for Example 7; and

FIG. 5 shows the color shift of solutions containing 0% to 100% HFCS after exposure to heat.

### DETAILED DESCRIPTION OF THE INVENTION

Liquid DPH medication can contain flavors, sweeteners, and optionally colorants to make it aesthetically pleasing. For instance, liquid ZzzQuil®, a medicine containing DPH that is distributed by Procter & Gamble®, contains colorants that include FD&C Blue No. 1 and FD&C Red No. 40, which give the medicine a pleasing purple color, and a flavoring system that contains HFCS. Currently, liquid ZzzQuil® is packaged in a translucent bottle with a UV-inhibitor to avoid degradation of the colorant and DPH. The United States Pharmacopeia (USP) requires liquid medications that contain DPH to be sold in a tight, light resistant container. In essence, this means that in order to comply with the USP, the primary package is light resistant, blocking UV light and visible light with a wavelength between 290-450 nm and/or a secondary package, such as a cardboard box, is provided. For instance, some liquid medications are sold in amber or opaque bottles and optionally a secondary package such as a cardboard box. In some examples, the primary packaging only blocks UV light

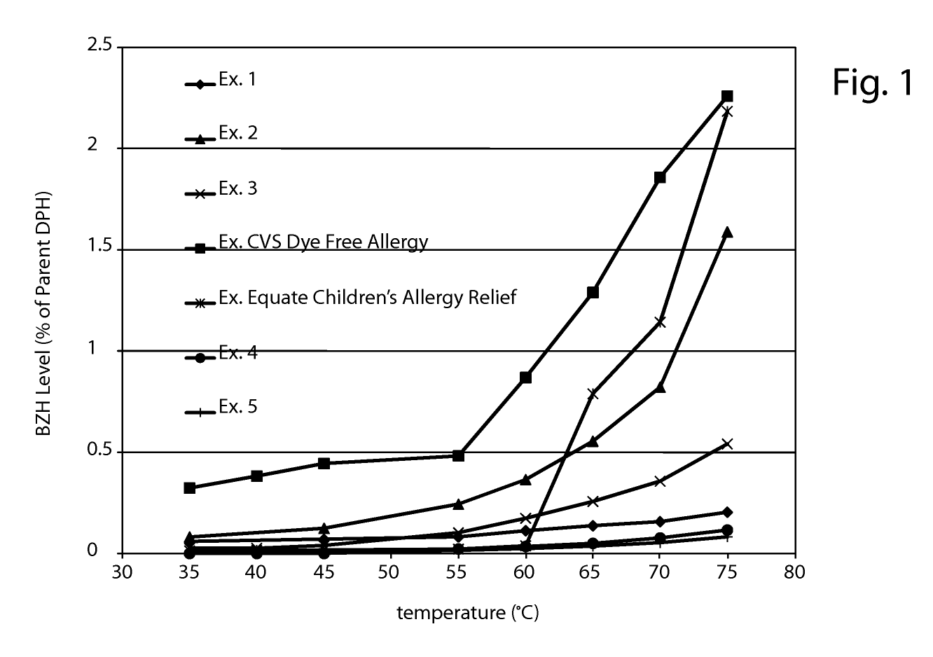

FIG. 1 shows the dependence of benzhydrol (BZH) formation as a function of temperature after 14 days for Examples 1-5 and two commercially available products. BZH is a known degradant of DPH and the more BZH that forms, the more DPH has degraded. Examples 1, 4, and 5 had the least BZH formation and are thus the most stable formulations. Examples 1, 4, and 5 have a pH of about 5.0, where the other formulations have a lower pH. Thus, pH can be a factor in the stability of DPH in solution. Furthermore, Ex. 2 contains HFCS, which can also contribute to an increased rate of degradant formation. The formulations that contain sorbitol degrade at a lower rate than the formulation with HFCS. Therefore, the most stable liquid medications can have a pH that is greater than about 4.5 and/or can be substantially free of HFCS.

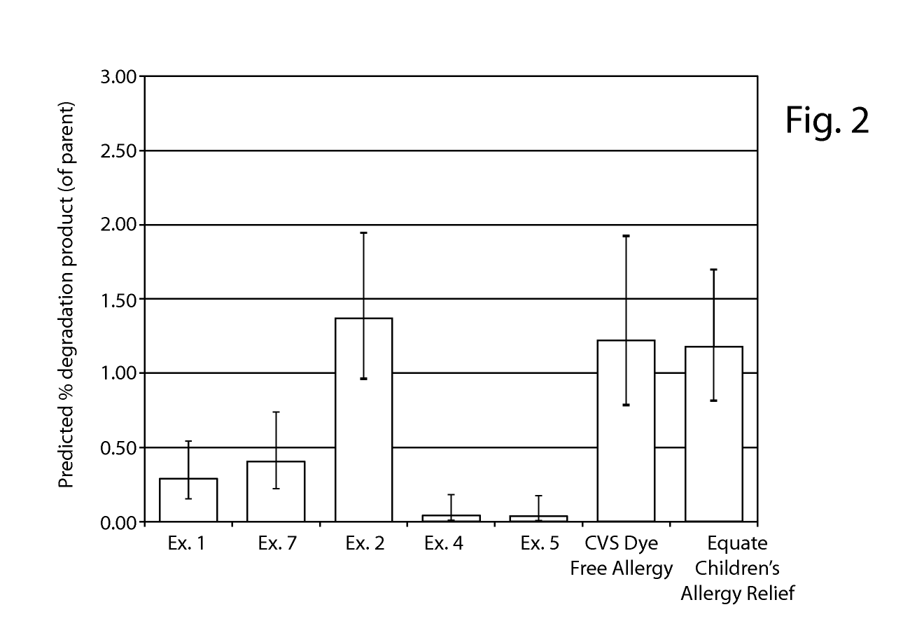

FIG. 2 shows a model for the predicted percent degradation in Examples 1, 2, 4, 5, and 7 and two currently available products after a period of 24 months at 25° C. Examples 4 and 5 were predicted to be most stable after two years.

Furthermore, the colorants, HFCS, and DPH in liquid medications can become unstable when subjected to extreme conditions, such as heat, which are often encountered during shipping, handling and storage of liquid medications. HFCS, can “brown” with the addition of heat thereby changing the color of the liquid medication. This browning can be apparent in any colored solution, however it is particularly apparent in liquid medications that are colorless.

The liquid medication can be stable when sold and stored in a colorless or colored translucent primary container, even without a primary container with a UV-inhibitor and/or a secondary container that blocks UV light. This can reduce packaging cost and can increase the aesthetic appearance of the product at the store shelf. A translucent bottle can also make it easier for the consumer to determine how much medication has been used.

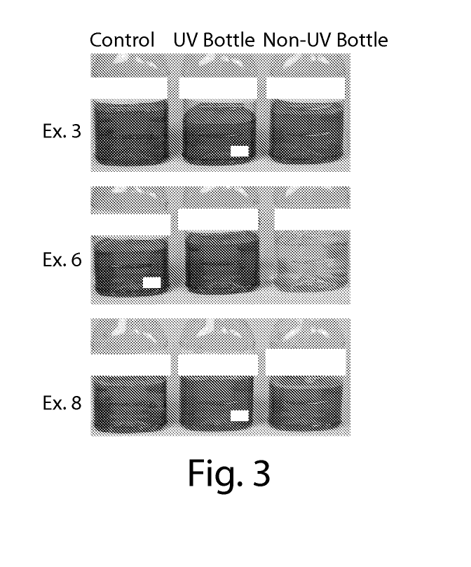

It was found that liquid medications comprising FD&C Blue No. 1 or FD&C Red No. 40 and HFCS had a color shift when exposed to UV light when stored in a standard bottle. Standard bottles are colorless, translucent bottles without a UV-inhibitor. Most significantly, the Blue No. 1 was photo-bleached by the UV light, while the Red No. 40 faded, as seen in FIG. 3. The liquid medications comprising Blue No. 1 or Red No. 40, DPH, and HFCS were exposed to the conditions described in the Photostability Testing, as described hereafter. Under these conditions, the DPH degraded and both the Blue No. 1 and Red No. 40 had a significant color shift.

Surprisingly, it was found that medications that were free of HFCS and formulated at a pH of about 5.0 were chemically stable, color stable, and physically stable even when stored in standard bottles without a UV-inhibitor. Furthermore, formulations that were formulated at a pH of about 5 and were substantially free of HFCS had greater color stability during the Photostability Testing, described hereafter, and fewer degradants formed during the Stability Prediction Method, described hereafter.

As used herein, “dose” refers to a volume of liquid medication containing an amount of a drug active suitable for administration on a single occasion, according to sound medical practice. A dose can be orally administered and is typically swallowed immediately. In one example, a dose can be about 30 mL, in another example about 25 mL, in another example about 20 mL, in another example about 15 mL, and in another example about 10 mL. The concentration of active ingredients can be adjusted to provide the proper doses of actives given the liquid dose size.

As used herein, “medication” refers to medications, such as pharmaceuticals, including prescription medications and/or over-the-counter medications (OTC). In one example, the medication is OTC.

FIG. 1 shows the dependence of BZH formation as a function of temperature after exposure to the Stability Prediction Method for 14 days, as described hereafter. Examples 1-5 and two commercial products were tested.

Example 1, which contains sorbitol and has a pH of about 5.0, Example 2, which contains HFCS and had a pH of about 4.0, Ex. 3, which contains sorbitol and has a pH of about 4.0, and Ex. 4 and Ex. 5, which both have a pH of about 5.0 and contain sorbitol.

The commercial products are CVS® Dye-Free Children's Allergy (Lot #21772, Expiration: April 2015) and Equate® Children's Allergy Medicine Allergy Relief (Lot #144984, Expiration: March 2015). Both CVS® and Equate® products are sold in secondary packaging, which is a cardboard box. The primary packaging for the Equate® product is a translucent bottle without a UV-inhibitor and CVS® product's primary package is a dark colored, opaque bottle. The CVS@ product contains the following ingredients: Active Ingredient (in Each 5 ml, 1 Teaspoon): Diphenhydramine HCl (12.5 mg). Inactive Ingredients: carboxymethylcellulose sodium, citric acid, flavors, glycerin, purified water, saccharin sodium, sodium benzoate, sodium citrate, and sorbitol solution. The Equate® product contains following ingredients: Active Ingredient (in Each 5 ml, 1 Teaspoon): Diphenhydramine HCl (12.5 mg). Inactive Ingredients: carboxymethylcellulose sodium, citric acid, flavors, glycerin, purified water, saccharin sodium, sodium benzoate, and sorbitol.

Examples 1, 4 and 5 had less BZH formation when compared to Examples 2 and 3. At the highest temperature, Example 2 had about an 8× increase in BZH as compared to Example 1 and 7. By plotting the degradant formation data in an Arrhenius plot, the Arrhenius parameters (energy of activation (Ea) and collision constant (A)) were determined. Using these parameters and modeling via the Arrhenius equation results in about a 2.4 to 3.6× increase in the formation rate at real-time stability conditions of 25° C. for Example 2 when compared to Examples 1 and 7. Thus, from a degradant formation perspective, DPH is substantially more stable in a formulation that has a pH of about 5.0, as Examples 1, 4, and 5 all have a pH of about 5.0.

The commercial products were the least stable. Both the CVS® and Equate® products had a pH of less than 5.0. The CVS® product has a pH of 3.67 and the Equate® product has a pH of 4.79. Furthermore, both the CVS® and Equate® products contained carboxymethylcellulose (CMC). While not wishing to be bound by theory, it is believed that CMC can also cause a DPH liquid medication to be less stable.

In one example, the liquid medication can be substantially free of CMC. As used herein, “substantially free of CMC” refers to less than about 0.33%, in another example less than about 0.1%, in another example less than about 0.05%, in another example less than about 0.01%, and in another example less than about 0.001%. In another example, the liquid medication can be free of CMC. It can be desirable for a formulation to be substantially free of CMC for stability reasons, as discussed above, and because consumers who are taking liquid medications containing DPH to treat itchiness, insomnia, motion sickness and/or extrapyramidal symptoms may desire a thinner solution, which does not provide as much throat coating as a thicker solution. In another example, the liquid medication can be substantially free or free of xanthan gum.

In one example, the liquid medication can have a viscosity of less than about 10 cP as determined by the Viscosity Test method described hereafter, in another example less than about 6 cP, in another example less than about 5.5 cP, in another example less than about 5 cP, in another example less than about 4.5 cP, in another example less than about 4 cP, in another example less than about 3.5 cP, in another example less than about 3 cP, and in another example less than about 2.5 cP. In another example, the liquid medication can have a viscosity from about 1 cP to about 10 cP as determined by the Viscosity Test method described hereafter, in another example from about 2 cP to about 7 cP, in another example from about 2.2 cP to about 5.25 cP, in another example from about 2.6 cP to about 4.75 cP, and in another example from about 2.75 cP to about 4.5 cP.

In one example, the liquid medication comprises less than about 2.3% of BZH based on parent DPH for 14 days at 75° C. according to the Stability Prediction Method, in another example less than about 2.25%, in another example less than about 2.2%, in another example less than about 2.18%, in another example less than about 1.6%, in another example less than about 1.5%, in another example less than about 1.4%, in another example less than about 1.2%, in another example less than about 1%, in another example less than about 0.8%, in another example less than about 0.5%, in another example less than about 0.25%, in another example less than about 0.2%, in another example less than about 0.15%, and in another example less than about 0.1%. In another example, the liquid medication comprises from about 0.05% to about 3% BZH based on parent DPH for 14 days at 75° C. according to the Stability Prediction Method, in another example from about 0.1% to about 2.3%, in another example from about 0.15% to about 2%, in another example from about 0.2% to about 1.5%, and in another example from about 0.5% to about 1%.

In one example, the liquid medication has a pH of greater than about 4.0, in another example greater than about 4.25, in another example greater than about 4.5, in another example greater than about 4.70, in another example greater than about 4.80, in another example greater than about 4.85, and in another example greater than about 4.90. In another example, the liquid medication has a pH from about 4.5 to about 7.0, in another example from about 4.80 to about 6.5, and in another method from about 4.80 to about 5.5. The pH is measured using the pH Test Method, described hereafter.

FIG. 2 shows a model for the predicted percent degradation in Examples 1, 2, 4, 5, and 7 and two currently available products over a period of 24 months at 25° C. These values are calculated using the Arrhenius equation and the parameters determined from an Arrhenius plot of the stability data from FIG. 1. Example 7 is not shown in FIG. 1, however when the Stability Prediction data for Example 7 is plotted in the same fashion as in FIG. 1 it approximately overlaps with the line for Example 1. The rate is calculated for 25° C. to determine the predicted percent degradation of the product (reported as % of BZH based on parent). Examples 4 and 5 had the lowest predicted degradation with a mean degradation of 0.04%. Examples 1 and 7 also had acceptable predicted degradation with a mean predicted degradation of 0.29% and 0.41%, respectively. The commercial products had mean predicted degradation of 1.22% and 1.18%, which is significantly higher than Examples 1, 7, 4 and 5. These low levels of predicted degradation rates indicate that the DPH is more stable and can result in a longer shelf life. In one example, the shelf life is greater than or equal to about 18 months, in another example greater than or equal about 2 years, in another example greater than or equal 2.5 years, and in another example greater than or equal to about 3 years.

In another example, the mean predicted degradation of the liquid medication (reported as % of BZH based on parent) over two years was less than about 3%, in another example less than about 2.5%, in another example less than about 2%, in another example less than about 1.8%, in another example less than about 1.7%, in another example less than about 1.5%, in another example less than about 1.35%, in another example less than about 1.25%, in another example less about 1.20%, in another example less than about 1.18%, in another example less than about 1.15%, in another example less than about 1.05%, in another example less than about 0.75%, in another example less than about 0.6%, in another example less than about 0.5%, in another example less than about 0.4%, in another example less than about 0.3%, in another example less than about 0.25%, in another example less than about 0.15%, in another example less than about 0.1%, in another example less than about 0.5%, and in another example less than about 0.02%. In one example the mean predicted degradation of the liquid medication (reported as % of BZH based on parent) is from about 0.001% to about 1.22%, in another example from about 0.01% to about 0.7%, in another example from about 0.02% to about 0.45%, and in another example from about 0.03% to about 0.30%.

Testing was also performed to determine the photostability of the Blue No. 1 and Red No. 40 in formulations that do and do not comprise HFCS. The Photostability Testing was done according to the International Conference on Harmonised (ICH) Tripartite Guideline Q1B entitled Stability Testing: Photostability Testing of New Drug Substances (dated Nov. 6, 1996) (referred to hereafter as “ICH Conditions”). The light source is an Atlas SUNTEST XLS+ (available from Atlas Material Testing Technology, Chicago, Ill.). The examples were tested by following the ICH Conditions, however the exposure time for each sample was five times longer (referred to hereafter as “ICH+ Conditions”).

Three aliquots were removed from each formulation, corresponding to Examples 3, 4 or 6, described hereafter, and each aliquot was placed into a control bottle, a bottle with a UV-inhibitor, or a standard bottle without a UV-inhibitor. All bottles were 6 oz. and made out of polyethylene terephthalate (PET) and had plastic screw top closures. The aliquots in the bottle with the UV-inhibitor and the standard bottle were subjected to the ICH+ Conditions.

The color shift was determined by a visual inspection and by the Colorimeter Method, as described hereafter.

FIG. 3 shows the color shift for Examples 3, 6, and 8 that were subjected to ICH+ Conditions. For Example 8, a formulation that does not contain HFCS, the dye system remained stable. For Examples 3 and 6, which both contain HFCS, there was more color fade than for the example without HFCS. In particular, the magnitude of the color shift in Example 6 is shown. Example 6 was completely photo-labile in a non-UV bottle and turned clear upon exposure to ICH+ Conditions. However, this loss of color was not observed in the UV-inhibitor containing bottle. Example 3, which comprises HFCS and Red No. 40 colorant, also experienced slight color fading after prolonged exposure periods and thus was noticeably different from the unexposed controls.

Samples of Examples 2, 3, 6, and 7, as described below, were subjected to a rigorous drug stability evaluation to understand DPH stability in different formulations, according to the Forced Degradation Stability Testing as described hereafter.

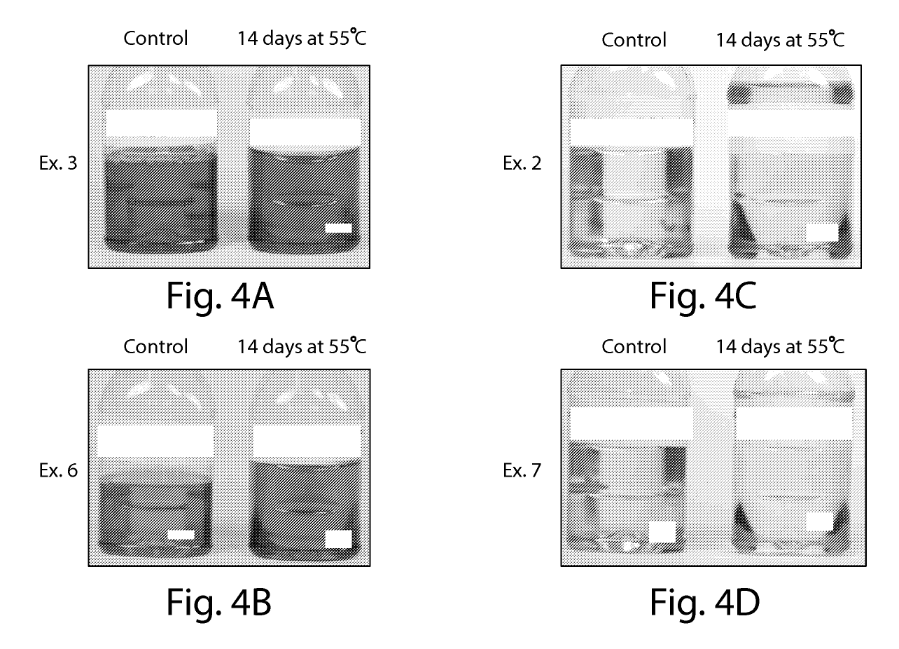

FIG. 4A shows the magnitude of the color shift for Example 3, which contains HFCS and Red No. 40. The heating causes the color to shift from red to an orange.

FIG. 4B shows the magnitude of the color shift for Example 6, which contains HFCS and FD&C Blue No. 1. The heating causes the blue liquid to change color to a deep turquoise.

FIG. 4C shows the magnitude of color shift for Example 2, which contains HFCS and no colorant. The heating caused the liquid to change to a yellow/brown color.

FIG. 4D shows the magnitude of color shift for Example 7, which contains a sorbitol sweetener instead of HFCS. The formulation with the sorbitol had no significant color change. Thus, formulations that do not comprise HFCS are more color stable during the conditions in the Forced Degradation Stability Testing, than formulations with HFCS.

The liquid medication can be color stable. In one example, the color change is not visually perceptible. As used herein, “visually perceptible” means that a human viewer can visually discern the color change with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

In another example, the color change can be determined by the Colorimeter Method, described hereafter.

Making a stable liquid medication with DPH and no colorant can be especially challenging. Not only do certain sweeteners, like HFCS, turn brown when exposed to light, like Example 2 in FIG. 4C, but DPH, and other actives and/or excipients, can precipitate out of solution. In some examples, this precipitate may not be noticeable to consumers if the liquid contains a colorant, especially if the colorant is a dark color like blue or purple, but can be more noticeable in a clear solution.

In one example, the DPH can form a co-crystal with saccharin and/or acesulfame potassium. This co-crystal precipitate can form in the bottles over time. In one example, the liquid medication may not contain acesulfame potassium and/or saccharin. In another example, a surfactant, such as Polyoxyl 40 stearate, maybe included to reduce co-crystal formation.

In one example, the liquid medication can be physically stable. In one example, a precipitate is not visually perceptible. In another example, the liquid medication can have a turbidity of less than about 10 NTUs, in another example less than about 1 NTUs, in another example less than about 0.5 NTUs, in another example less than about 0.25 NTUs, in another example less than 0.1 NTUs, and in another example less than 0.05 NTUs.

A dose of liquid medication can be from about 5 mL to about 75 mL, in another example from about 15 mL to about 50 mL, in another example from about 25 mL to about 40 mL, and in another example from about 28 mL to about 35 mL. In one example, a dose of the liquid medication is about 30 mL, in another example about 20 mL, and in another example about 15 mL. In one example, the dose is intended to be administered every 24 hours. In another example, the dose is intended to be administered every 4 hours or every 6 hours.

In one example, the liquid medication comprises about 50 mg DPH per dose and is intended for consumption by adults and children 12 years and over. In another example, the medication comprises 25 mg DPH per dose and can be taken by anyone 6 years and over. In another example, the medication can contain 25 mg DPH per dose and can be taken by adults and children 12 years and over. In another example, the medication comprises 12.5 mg DPH per dose and can be taken by children ages 6 to 11.

In one example, the liquid medication can be stable and can be sold and/or stored in a translucent primary container without a UV-inhibitor and without a secondary container that prevents light from passing through. In another example, the liquid medication can be stable and can be sold and/or stored in a translucent primary container that can block UV light, but does not block visible light and does not include a secondary container that prevents light from passing through. In another example, the primary container can be translucent and/or colorless. The primary container can be made out of any suitable material. Non-limiting examples of suitable materials for the primary container can include polyethylene terephthalate (PET), Glycol-modified Polyethylene Terephthalate (PETG), Oriented Polypropylene (OPP), Polyvinylchloride (PVC), Polyvinylidene Chloride (PVDC), Nylon, Polyethylene Terphthalate Polyester (PETP), Polyphene, and combinations thereof. In one example, the container can be made out of PET.

The liquid medication can comprise a flavoring system. The flavoring system can comprise sweeteners, sensates, flavoring ingredients, salivating agents and combinations thereof.

The medications can comprise a sweetener to provide sweetness and taste masking of the DPH as well as any additional actives that may be present. In one example, the medication comprises from about 5% to about 45% sweetener, in another example from about 10% to about 40% sweetener, in another example from about 15% to about 35% sweetener, and in another example from about 20% to about 30% sweetener. Non-limiting examples of sweeteners can include nutritive sweeteners, sugar alcohols, synthetic sugars, high intensity natural sweeteners, and combinations thereof.

Non-limiting examples of nutritive sweeteners can include fructose, galactose, and combinations thereof.

In one example, the liquid medication is substantially free of reducing sugars. Non-limiting examples of reducing sugars can include HFCS, glucose, fructose, and combinations thereof. In another example, the liquid medication is substantially free of sucrose, including liquid sucrose, because sucrose can hydrolyze to its constituent sugars, namely glucose and fructose.

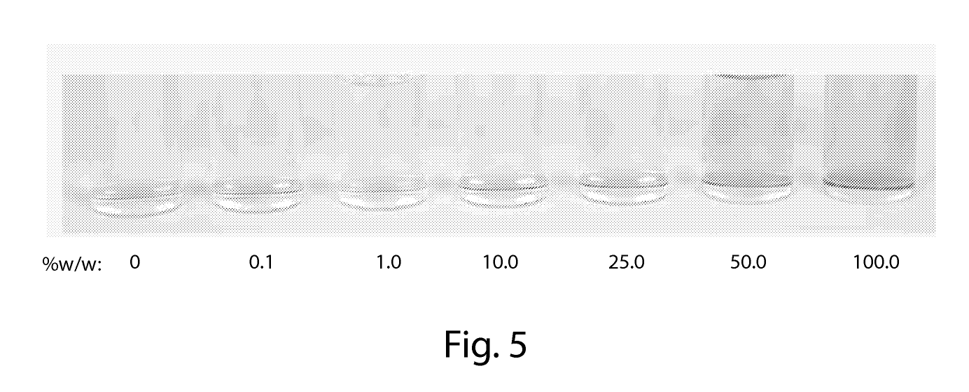

FIG. 5 shows the color shift of solutions containing 0% to 100% HFCS. The HFCS was exposed to the conditions in the Forced Degradation Stability Testing as described hereafter, except the solution was held at 75° C. for 24 hours. A solution that contains HFCS that is discolored, is not acceptable to consumers. The samples with 25%, 50%, and 100% HFCS are noticeably discolored. However, the sample with 10% HFCS may not be acceptable to consumers, as the color change may be visually perceptible, especially if it is placed on a shelf with liquid medications that are not discolored. However, the color change, if any, for the examples with 1% and 0.1% HFCS is not visually perceptible, even when compared to the sample with 0% HFCS. As used herein, “substantially free of HFCS” refers to less than about 10% HFCS, in another example less than about 7% HFCS, in another example less than about 5% HFCS, in another example less than about 3% HFCS, in another example less than about 1% HFCS, in another example less than about 0.5% HFCS, in another example less than about 0.25% HFCS, in another example less than about 0.1% HFCS, and in another example less than about 0.01% HFCS. In another example, the liquid medication can be free of HFCS.

Non-limiting examples of sugar alcohols can include xylitol, sorbitol, mannitol, maltitol, lactitol, isomalt, erthritol, glycerin, and combinations thereof. In one example, the sugar alcohol can be sorbitol. In one example the medication can comprise from about 10% to about 40% sugar alcohol, in another example from about 20% to about 35% sugar alcohol, and in another example about 25% to about 31% sugar alcohol. In another example the medication can comprise from about 1% to about 30% sugar alcohol, in another example 5% to about 25%, in another example from about 10% to about 20%, and in another example from about 12% to about 16%.

In another example, the medication can contain glycerin. Glycerin is a viscous liquid and can improve the mouthfeel of the liquid medication, which can be helpful especially in medications that are substantially free of HFCS. In one example, the liquid medication contains from about 1% to about 20% glycerin, in another example from about 3% to about 15%, and in another example from about 5% to about 10%.

Non-limiting examples of synthetic sweeteners can include sodium saccharin, acesulfame potassium, sucralose, aspartame, monoammonium glycyrrhizinate, neohesperidin dihydrochalcone, thaumatin, neotame, cyclamates, and mixtures thereof. In one example the medication can comprise from about 0.01% to about 0.5% synthetic sweetener, in another example from about 0.1% to about 0.3%, and in another example about 0.15% to about 0.25%.

In one example, DPH is the only drug active in the liquid medication. The liquid medication can be used as a sleep-aid or to help treat allergic symptoms.

In another example, the liquid medication can contain drug actives in addition to DPH. In one example, the additional drug active can be a pain reliever. In one example, the liquid medication can be taken at nighttime. In another example, the additional drug active can be selected from the group consisting of loratadine, oxymetazoline, pseudophedrine, phenylephrine, pseudophedrine, levmetamfetamine, and combinations thereof.

Non-limiting examples of pain relievers can include acetaminophen (APAP), ibuprofen, ketoprofen, diclofenac, naproxen, aspirin, and combinations thereof. In one example the liquid medication can comprise from about 0.5% to about 3.5% pain reliever, in another example from about 1% to about 3% pain reliever, and in another example from about 1.5% to about 2% pain reliever. In one example the pain relievers can include APAP, ibuprofen, naproxen, or combinations thereof. In one example a dose can comprise 325 mg to 500 mg APAP, in another example 200 mg ibuprofen, and in another example 200 mg naproxen.

The present liquid components typically comprise a solvent. A solvent can be used to dissolve the DPH, flavoring system, and/or other active(s) into solution.

Non-limiting examples of solvents can include water, propylene glycol, polyethylene glycol, ethanol, and mixtures thereof. In one example the medication comprises from about 40% to about 95% solvent, in another example from about 50% to about 80% solvent, and in another example from about 55% to about 60% solvent, and in another example from about 68% solvent to about 72% solvent.

In one example, the medication can contain water and propylene glycol. In one example, the medication comprises from about 15% to about 80% water, in another example from about 25% to about 75% water, in another example from about 40% to about 70% water, in another example from about 35% to about 45% water, and in another example from about 57% to about 66% water. In another example, the medication can comprise from about 1% to about 10% propylene glycol, in another example from about 2% to about 8% propylene glycol, and in another example from about 3% to about 6% propylene glycol. In another example, the medication can comprise from about 1% to about 15% ethanol, in another example from about 3% to about 12% ethanol, and in another example from about 6% to about 10% ethanol.

In one example, the medication can contain a buffer. The buffer can help maintain a constant pH within the liquid medication. In one example the liquid medication can contain from about 0.05% to about 2% buffer, in another example from about 0.1% to about 1% buffer, in another example from about 0.15% to about 1% buffer, and in another example from about 0.30% to about 0.50% buffer. Buffers can include acetate buffers, citrate buffers, and phosphate buffers. Non-limiting examples of buffers can include acetic acid, sodium acetate, citric acid, sodium citrate, monobasic sodium phosphate, dibasic sodium phosphate, sodium carbonate, sodium bicarbonate, succinic acid, sodium succinate, potassium dihydrogen phosphate, and phosphoric acid.

In one example, the medication can contain a preservative. In one example the liquid medication can contain from about 0.01% to about 1% preservative, in another example from about 0.05% to about 0.5% preservative, in another example from about 0.07% to about 0.3% preservative, and in another example from about 0.08% to about 0.15% preservative. Non-limiting examples of preservatives can include benzalkonium chloride, ethylenediaminetetraacetic acid (EDTA), benzyl alcohol, potassium sorbate, parabens, benzoic acid, sodium benzoate, and mixtures thereof.

In one example, the medication can contain a thickener. In one example the liquid medication can contain from 0.01% to 3% thickener, in another example 0.05% to 1.5% thickener, in another example 0.1% to 0.75% thickener, and in another example 0.12% to 0.3% thickener. Non-limiting examples of thickeners can include xanthan gum, carrageenan, polyacrylic acid, polyvinylpyrrolidone, cellulosic polymers including CMC, hydroxethylcellulose, hydroxymethylcellulose, and hydroxypropylmethylcellulose, and combinations thereof. In one example, the medication may not comprise a thickener.

The liquid medication can be any color. Non-limiting examples of colors can include red, green, amber, orange, yellow, blue, pink, purple, violet, turquoise, and combinations thereof. In one example, the medication can be purple. In another example, the medication can be red and in another example, the medication can be blue. In one example, the liquid medication can be substantially free of dye and can be colorless.

The medication can also comprise a dye that can provide the color. Non-limiting examples dyes that may be used in the present invention include FD&C blue #1, FD&C blue #2, D&C blue #4, D&C blue #9, FD&C green #3, D&C green #5, D&C green #6, D&C green #8, D&C orange #4, D&C orange #5, D&C orange #10, D&C orange #11, FD&C red #3, FD&C red #4, D&C red #6, D&C red #7, D&C red #17, D&C red #21, D&C red #22, D&C red #27, D&C red #28, D&C red #30, D&C red #31, D&C red #33, D&C red #34, D&C red #36, D&C red #39, FD&C red #40, D&C violet #2, FD&C yellow #5, FD&C yellow #6, D&C yellow #7, Ext. D&C yellow #7, D&C yellow #8, D&C yellow #10, D&C yellow #11, and combinations thereof. In one example, the medication comprises from about 0.001% to about 0.1% dye, in another example from about 0.002% to about 0.05% dye, and in another example form about 0.003% to about 0.01% dye.

In one example, the liquid medication can contain a color and the color can be stable under ICH Conditions in a primary container without a UV-inhibitor. In one example color stable means that no color change is visually perceptible. In one example, when the L dimension is measured soon after the sample is made and then after the sample has been stored at ICH Conditions in a primary container without a UV-inhibitor, the L dimension changes less than about ±60%, in another example less than about ±55%, in another example less than about ±50%, in another example less than about ±45%, in another example less than about ±40%, and in another example less than about ±30%. In one example, when the a dimension is measured soon after the sample is made and then after the sample has been stored at ICH Conditions in a primary container without a UV-inhibitor, the a dimension changes less than about ±40%, in another example less than about ±35%, in another example less than about ±30%, in another example less than about ±25%, and in another example less than about ±20%. In one example, when the b dimension is measured soon after the sample is made and then after the sample has been stored at ICH Conditions in a primary container without a UV-inhibitor, the b dimension changes less than about ±40%, in another example less than about ±35%, in another example less than about ±30%, in another example less than about ±25%, and in another example less than about ±20%. In another example, the L dimension, after ICH Conditions in a primary container without a UV-inhibitor can be from about 20 to about 80, in another example from about 25 to about 70, in another example from about 30 to about 60, in another example from about 35 to about 50, and in another example from about 42 to about 48. The Hunter L-a-b dimensions can be determined according to the Colorimeter Method, described hereafter.

In one example, the liquid medication can be substantially free or free of alcohol, including but not limited to ethanol. In another example, the liquid medication can be substantially free of artificial flavors. In another example, the liquid medication can be substantially free of artificial sweeteners. In another example, the liquid medication can be substantially free of artificial dyes. In another example, the liquid medication can be substantially free of artificial preservatives.

In one example, the liquid medication can be a solution. In one example, the solution can be homogeneous and the excipients including the flavoring system and all actives can be dissolved. In another example, the liquid medication can be a suspension or it can be a colloid. In another example, the liquid medication is not a suspension. In another example, the liquid medication may not be a colloid.

### Examples

| Ingredient | Ex. 1 | Ex. 2 | Ex. 3 | Ex. 4 | Ex. 5 |
|---|---|---|---|---|---|
| Target pH | 5.0 | 4.0 | 4.0 | 5.0 | 5.0 |
| Diphenhydramine HCl | 0.15% | 0.15% | 0.15% | 0.16% | 0.16% |
| Propylene Glycol | 5.00% | 5.00% | 5.00% | 5.00% | 5.00% |
| Alcohol 95% USP | 7.84% | 7.55% | 7.84% | 8.00% | 8.00% |
| ethanol | | | | | |
| Flavor | 0.25% | 0.20% | 0.25% | 0.25% | 0.25% |
| Water Purified | 46.05% | 45.10% | 46.03% | 65.82% | 57.82% |
| Sodium Citrate Dihydrate | 0.36% | 0.19% | 0.19% | 0.36% | 0.36% |
| Citric Acid Anhydrous | 0.16% | 0.35% | 0.35% | 0.16% | 0.16% |
| Polyoxl 40 Stearate | 0.05% | 0.05% | 0.05% | 0.05% | 0.05% |
| Sodium Saccharin USP | 0.035% | 0.07% | 0.035% | 0.11% | 0.07% |
| Sodium Benzoate NF, FCC | 0.10% | 0.10% | 0.10% | 0.10% | 0.10% |
| Red No. 40 | 0.0040% | 0.00% | 0.0040% | 0.00% | 0.00% |
| FD&C Blue No. 1 | 0.0010% | 0.00% | 0.001% | 0.00% | 0.00% |
| High Fructose Corn Syrup | 0.00% | 41.24% | 0.00% | 0.00% | 0.00% |
| Sorbitol Solution 70% | 40.00% | 0.00% | 40.00% | 20.00% | 20.00% |
| Sucralose | 0.00% | 0.00% | 0.00% | 0.00% | 0.03% |
| Glycerin | 0.00% | 0.00% | 0.00% | 0.00% | 8.00% |

| Ingredient | Ex. 6 | Ex. 7 | Ex. 8 |
|---|---|---|---|
| Target pH | 4.0 | 5.0 | 5.0 |
| Diphenhydramine HCl | 0.15% | 0.15% | 0.15% |
| Propylene Glycol | 5.00% | 5.00% | 5.00% |
| Alcohol 95% USP | 7.55% | 7.84% | 7.84% |
| ethanol | | | |
| Flavor | 0.20% | 0.25% | 0.25% |
| Water Purified | 45.10% | 46.05% | 46.05% |
| Sodium Citrate Dihydrate | 0.19% | 0.36% | 0.36% |
| Citric Acid Anhydrous | 0.35% | 0.16% | 0.16% |
| Polyoxl 40 Stearate | 0.05% | 0.050% | 0.050% |
| Sodium Saccharin USP | 0.07% | 0.035% | 0.035% |
| Sodium Benzoate NF, FCC | 0.10% | 0.10% | 0.10% |
| Red No. 40 | 0.00% | 0.00% | 0.00% |
| FD&C Blue No. 1 | 0.0010% | 0.00% | 0.0010% |
| HFCS | 41.24% | 0.00% | 0.00% |
| Sorbitol Solution 70% | 0.00% | 40.00% | 40.00% |

Examples 1-8 were made as follows. First, a glycol premix was made by putting propylene glycol in a container and beginning agitation. Then DPH, ethanol, and the flavoring were added and mixed until all of the components were fully dissolved to form the glycol premix.

Then, a main mix was made by adding purified water to a container and beginning agitation. Then the buffer salts, which included sodium citrate dehydrate and citric acid, and surfactant, which included polyoxl 40 stearate, were added and mixed until dissolved. Then the sodium saccharin, sucralose (if present), colors, which included FD&C Red #40 and FD&C Blue #1, and sodium benzoate were added and mixed until dissolved to make the main mix.

Next, the glycol premix was added to the premix and the glycol premix vessel was rinsed with about 5 or 6 mL purified water and it was added to the main mix. Then the HFCS, sorbitol, and/or glycerin were added to the mixture and it was mixed until the solution was fully homogeneous. Finally, the pH was adjusted with citric acid and/or sodium citrate to achieve the target pH.

### Test Methods

### Colorimeter Method

The Colorimeter method is performed using a Color-view Spectrophotometer, Model 9000 (available from HunterLab, Reston, Va.). Using standard tiles, follow calibration protocol found in the manufacturer's instruction manual. After calibration, load sample into the petri dish.

After calibration, load 10 mL of the sample into the petri dish. Pour the liquid into the petri dish slowly to avoid creating bubbles. Make certain the entire bottom surface of the dish is covered by sample.

Place sample over the aperture so it is well seated and covers the entire opening. Cover the closed petri dish with a white background (paper or white standard tile) to prevent interference from ceiling light.

Immediately measure the sample. (If the sample is allowed to sit on the lighted port before the color is measured, it may result in excess photo degradation). The Hunter L-a-b reading should be displayed and recorded.

### Forced Degradation Stability Testing

A sample of each example was put into a standard 6 oz. polyethylene terephthalate (PET) bottle with plastic screw top closures. The samples were then placed in an FP 4000 Material Test Chamber with Mechanical Convection (available from Binder Inc., Bohemia, N.Y.). The samples were left in the oven for 14 days at 55° C. at ambient pressure. Then, the samples were removed for visual inspection and color evaluation by a colorimeter. The control samples were left at ambient temperature and pressure and stored in the dark.

### HPLC-UV Assay

This method is applicable for the determination of DPH and degradation products of DPH in respiratory liquid formulations. The sample is analyzed by HPLC using a C18 column with trifluoroacetic acid (TFA) and acetonitrile (ACN) mobile phases and a single point external standard for quantification. Detection is by UV absorbance at 225 nm with detector response measured by peak area.

Sample Preparation (Results Reported in % w/w)

Tare an appropriate volumetric flask. Transfer a sample of the liquid medication into the flask and record the weight to the nearest 0.1 mg. Dilute the liquid medication in the volumetric flask and Q.S. to volume with water and mix thoroughly. Filter the liquid medication and the water with the aid of a disposable syringe and a syringe filter into an injection vial and cap, to form the Sample Preparation. Record the volume (mL) and weight (g) of the Sample Preparation for use in the calculation below.

Stock Standard Solution Preparation

Depending on the concentration of the sample at t=0 and the weight of the Sample Preparation, weigh an appropriate amount of DPH reference standard to the nearest 0.1 mg and quantitatively transfer to a volumetric flask using 0.1% v/v phosphoric acid. Add water to volume and mix thoroughly ensuring that all standards have dissolved, to form the stock Standard. Record the flask volume used (mL) and the weight (g) of the Stock Standard.

Working Standard Solution Preparation

Dilute the Stock Standard to the target DPH concentration found in the prepared sample. Pipette 10.0 mL of the stock standard solution into a 100 mL volumetric flask. Add 0.1% v/v phosphoric acid to volume and mix thoroughly. Record the flask volume used (mL) for working standard preparation and the dilution factor.

Mobile Phase Preparation

Next, prepare the aqueous and organic mobile phase components, Mobile Phase A and B, respectively. To prepare Mobile Phase A, add 1 mL of TFA per 1 L of purified water. For Mobile Phase B, use 100% ACN. Mobile Phase A and B will be used to perform the reverse-phase gradient chromatography as described in USP Chapter <621> and the Chromatographic Conditions described below.

Chromatographic Conditions

The Waters XBridge™ reverse-phase HPLC columns (available from Waters Corporation, Milford, Mass.) are equipped with a 4.6×150 mm column that contains a 3.5 μm C18 packing material. The column temperature is 40° C. with the flow rate at 1.0 mL/min and the detector wavelength at 275 nm. The sample injection volume is 50 μL. Certain conditions such as the column temperature, flow rate, and mobile phase reagent ratio may be altered or changed provided that adequate resolution and sensitivity are obtained per USP Chapter <621> and System Suitability criteria are met.

System Suitability

For system suitability, inject and chromatograph the Working Standard until system suitability is achieved with five successive injections. The system suitability is the % RSD (Relative Standard Deviation) for the peak areas and the retention times for DPH should be 2.0% or less. Also the peak tailing for DPH should be 2.5 or less. Peak retention order is DPH followed by BZH.

Next, inject 10 μL of the Sample Preparation and chromatograph and then inject 10 μL of the Working Standard, this is the bracket standard.

Then, inject 10 μL of the Sample Preparation and chromatograph. Repeat this step up with up to six samples before injecting 10 μL of the Working Standard.

Calculations

DPH or BZH (% w/w)=(W_{S}/V_{F1})*(P/100)*(DF)*(A₂/A₁)*(V_{F2}/WP)*(100)

Where W_{S} in the weight of the DPH or BZH reference standard (g), V_{F1} is the volume of the flack used to prepare the stock standard (mL), P is the purity of the reference standard in %, DF is the dilution factor from preparation of the working standard, A₂ is the chromatographic response of the sample, A₁ is the average chromatographic response of the reference standards, V_{F2} is the flask volume for preparation of the sample and WP is the weight of the product (g).

BZH (% of parent DPH)=((% w/w BZH)/(% w/w DPH dose))*(100)

### pH Test Method

First, calibrate the Thermo Scientific Orion 320 pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 7 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 4 buffer and wait until the pH icon stops flashing and press the measure button. Rinse the electrode with distilled water and carefully wipe with a scientific cleaning wipe. Now the pH meter is calibrated and can be used to test the pH of a solution.

The pH of the liquid medication is measured using the calibrated pH meter at ambient temperature.

### Stability Prediction Method

The formation of a known DPH degradant, BZH, was determined by exposing aliquots comprising DPH to temperatures of 35° C., 45° C., 55° C., 60° C., 65° C., 70° C., and 75° C. for 3, 7, and 14 days. At each interval, the aliquots, as well as an unexposed control, were tested for the formation of BZH. BZH levels were determined by HPLC-UV assay with their formation calculated as % w/w and reported as % of Parent DPH. The % BZH level based on parent DPH at 75° C. after 14 days can be used as an estimate of stability.

A prediction of the % of BZH based on parent at 25° C. is determined using the Arrhenius relationship. The % w/w BZH was determined from HPLC-UV Assay, as described herein. Then, the rates of formation (k) for each temperature were determined against % w/w BZH values of an unexposed control sample. These formation amounts were then converted into units suitable for an Arrhenius plot, where Arrhenius parameters were derived using standard linear regression. The percent predicted BZH formation for each sample was accomplished using the calculated rate constant from the standard Arrhenius equation with accelerated stability conditions (25° C.) for temperature.

Predicted rates of formation were then calculated using the energy of activation and collision constant (A) determined from Arrhenius plots of the temperature stability data for input into the Arrhenius equation using real-time temperature. Additional information regarding use of the Arrhenius equation on drug stability can be found in Yoshioka, Sumie and J. Stella Valentino, Stability Drugs and Dosage of Forms, New York: Kluwer Academic, 2000 (see Chapter 2).

### Viscosity Test Method

The viscosity of the liquid medication can be measured using a digital Brookfield Viscometer (model RVDVII) with a CPE-41 spindle with temperature control. First, allow the samples and standards to equilibrate at room temperature prior to analysis. Calibrate the viscometer as disclosed in the operator's manual and check the viscosity using a standard. The viscosity is measured at 25° C.±0.5° C., with a 1 mm gap (distance between the rotating spindle and the wall of the RVDVII), at a shear rate of between 5 and 10 RPM (rotations per minute). Each measurement is taken for a period of two minutes to allow for the collection of enough data points to determine the average viscosity of the product (i.e. the spindle rotates at 1 rpm for 2 minutes).

Values disclosed herein as ends of ranges are not to be understood as being strictly limited to the exact numerical values recited. Instead, unless otherwise specified, each numerical range is intended to mean both the recited values and any integers within the range. For example a range disclosed as “1 to 10” is intended to mean “1, 2, 3, 4, 5, 6, 7, 8, 9, 10.”

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A chemically stable liquid medication comprising diphenhydramine hydrochloride and a pH of greater than about 4.5 and wherein the liquid medication comprises less than about 1.5% of BZH based on parent DPH after 14 days at 75° C. according to the Stability Prediction Method and wherein the liquid medication is adapted for consumption by adults and children 12 years and over.

2. The liquid medication of claim 1 wherein the pH is greater than about 4.90.

3. The liquid medication of claim 1 wherein the medication is substantially free of high fructose corn syrup.

4. The liquid medication of claim 1 wherein the medication is substantially free of carboxymethyl cellulose.

5. The liquid medication of claim 1 wherein the medication has a viscosity from about 2.2 cP to about 5.25 cP.

6. The liquid medication of claim 1 wherein the medication comprises less than about 1% of BZH based on parent DPH after 14 days at 75° C. according to the Stability Prediction Method.

7. The liquid medication of claim 1 wherein the medication comprises less than about 0.5% of BZH based on parent DPH after 14 days at 75° C. according to the Stability Prediction Method.

8. The liquid medication of claim 1 further comprising glycerin.

9. The liquid medication of claim 1 further comprising sorbitol

10. A color stable liquid medication comprising diphenydramine and a pH of greater than about 4.0 wherein a color change is not visually perceptible and wherein the medication is substantially free of high fructose corn syrup.

11. The liquid medication of claim 10 wherein the pH is greater than about 4.8.

12. The liquid medication of claim 10 wherein the medication is chemically stable and wherein the mean predicted degradation of the product over two years at 25° C. is less than about 1.15%.

13. The liquid medication of claim 10 wherein the viscosity of less than about 6 cP.

14. The liquid medication of claim 10 wherein the medication is physically stable and wherein a precipitate is not visually perceptible.

15. A chemically stable liquid medication comprising diphenhydramine hydrochloride and a pH of greater than about 4.5 and wherein the liquid medication is contained in a translucent container and wherein the container does not have a UV-inhibitor and no secondary container is required for stability.

16. The liquid medication of claim 15 wherein the container is colorless.

17. The liquid medication of claim 15 wherein the medication is substantially free of high fructose corn syrup.

18. The liquid medication of claim 15 wherein the medication is substantially free of carboxymethyl cellulose.

19. The liquid medication of claim 15 wherein the medication comprises less than about 1% of BZH based on parent DPH after 14 days at 75° C. according to the Stability Prediction Method.

20. The liquid medication of claim 15 further comprising a colorant.
